(19) United States
(12) Patent Application Publication
(10) Pub. No.: US 2011/0245567 A1
(43) Pub. Date: Oct. 6, 2011

(54) PROCESS FOR OLIGOMERIZING DILUTE ETHYLENE

(75) Inventors: Joao Jorge da Silva Ferreira Alves, Arlington Heights, IL (US); Christopher P. Nicholas, Evanston, IL (US)

(73) Assignee: UOP LLC, Des Plaines, IL (US)

(21) Appl. No.: 12/751,320

(22) Filed: Mar. 31, 2010

(51) Int. Cl. C07C 2/02

(52) U.S. Cl. 585/518

(57) ABSTRACT

The process converts ethylene in a dilute ethylene stream that may be derived from an FCC product to heavier hydrocarbons. The oligomerization reactor is in communication between a primary absorber column and a secondary absorber column in an FCC product recovery section. The oligomerization catalyst may have a low silica base with a Group VIIIB metal and operate at low pressure without excessive deactivation. The catalyst is resistant to feed impurities such as hydrogen sulfide, carbon oxides, hydrogen and ammonia. At least 40 wt-% of the ethylene in the dilute ethylene stream can be converted to heavier hydrocarbons.

### BACKGROUND OF THE INVENTION

The field of the invention is a process for converting diluted ethylene in a hydrocarbon stream to heavier hydrocarbons. These heavier hydrocarbons may be used as motor fuels.

Dry gas is the common name for the off-gas stream from a fluid catalytic cracking unit that contains all the gases with boiling points lower than ethane. The off-gas stream is compressed to remove as much of the C₃ and C₄ gases as possible. Sulfur is also largely absorbed from the off-gas stream in a scrubber that utilizes an amine absorbent. The remaining stream is known as the FCC dry gas. A typical dry gas stream contains 5 to 50 wt-% ethylene, 10 to 20 wt-% ethane, 5 to 20 wt-% hydrogen, 5 to 20 wt-% nitrogen, about 0.05 to about 5.0 wt-% of carbon monoxide and about 0.1 to about 5.0 wt-% of carbon dioxide and less than 0.01 wt-% hydrogen sulfide and ammonia with the balance being methane.

Currently, the FCC dry gas stream is burned as fuel gas. An FCC unit that processes 7,949 kiloliters (50,000 barrels) per day will generate and burn about 181,000 kg (200 tons) of dry gas containing about 36,000 kg (40 tons) of ethylene as fuel per day. Because a large price difference exists between fuel gas and motor fuel products or pure ethylene it would appear economically advantageous to attempt to recover this ethylene. However, the dry gas stream contains impurities that can poison oligomerization catalyst and is so dilute in ethylene that its recovery is not economically justified by gas recovery systems.

The oligomerization of concentrated ethylene streams to liquid products is a known technology. However, oligomerization typically involves the use of propylene or butylene particularly from liquefied petroleum gas (LPG) or dehydrogenated feedstocks to make gasoline range olefins. Ethylene is little used as an oligomerization feedstock because of its much lower reactivity.

The economic opportunity created by the recovery of ethylene from dry gas is significant, creating a need for utilization of dilute ethylene in refinery streams.

### SUMMARY OF THE INVENTION

In an exemplary aspect, the process of the present invention comprises contacting a first FCC product stream with a second FCC product stream to recover a dilute ethylene stream. The dilute ethylene stream is contacted with an oligomerization catalyst to provide an oligomerization product comprising ethylene oligomers. The oligomerization product is contacted with a third FCC product stream.

In a second exemplary aspect, the process of the present invention comprises taking a first FCC product stream comprising between about 5 and about 50 wt-% ethylene and at least one impurity selected from the group consisting of at least about 0.05 wt-% carbon monoxide, at least about 1 wppm hydrogen sulfide, at least about 1 wppm ammonia, at least about 5 wt-% hydrogen and at least about 0.1 wt-% carbon dioxide. A portion of the first FCC product stream is contacted with an oligomerization catalyst to provide an oligomerization product comprising ethylene oligomers. The oligomerization product is then contacted with another FCC product stream.

In a third exemplary aspect, the process of the present invention comprises contacting a first FCC product stream with a second FCC product stream to recover a dilute ethylene stream. The dilute ethylene stream is contacted with an oligomerization catalyst having a base with a silicon-to-aluminum ratio of at least 20 and including a Group VIIIB metal to provide an oligomerization product stream comprising ethylene oligomers. The oligomerization product stream is contacted with a third FCC product stream to absorb ethylene oligomers. The third FCC product stream and absorbed ethylene oligomers are delivered to an FCC main fractionation column.

Advantageously, the process can enable utilization of ethylene in a dilute stream and in the presence of feed impurities that can be catalyst poisons.

Additional features and advantages of the invention will be apparent from the description of the invention, the FIGURES and claims provided herein.

### BRIEF DESCRIPTION OF THE DRAWINGS

FIG. 1 is a schematic drawing of an FCC unit and an FCC product recovery section.

FIG. 2 is an alternative schematic drawing of an FCC unit and an FCC product recovery section.

### DEFINITIONS

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “column” means a distillation column or columns for separating one or more components of different volatilities which may have a reboiler on its bottom and a condenser on its overhead. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the outlet of the column. The bottom temperature is the liquid bottom outlet temperature. Overhead lines and bottoms lines refer to the net lines from the column downstream of the reflux or reboil to the column.

As used herein, the term “a component-rich stream” means that the rich stream coming out of a vessel has a greater concentration of the component than the feed to the vessel.

### DETAILED DESCRIPTION

We have found that ethylene in dilute ethylene streams, such as an FCC dry gas stream, can be catalytically oligomerized to relatively low molecular weight dimer and trimer hydrocarbons with a Group VIIIB metal on a low acidity amorphous silica catalyst at lower pressure. In an embodiment, the oligomerization can be performed in a FCC product recovery section at pressure typical in the product recovery section. The absence of higher compression requirements to control deactivation allows placement of the oligomerization reactor upstream of the secondary absorber column. Oligomerization product can then be processed in the FCC product recovery section without needing additional equipment. The oligomers can be separated and processed in the FCC product recovery section while the unconverted ethylene and lighter gas can be processed as is typical in a refinery. The unconverted gas may be burned as fuel gas, but with the more valuable ethylene removed and processed as heavier hydrocarbons.

The present invention may be applied to any hydrocarbon stream containing ethylene and, preferably, a dilute proportion of ethylene. A suitable, dilute ethylene stream may typically comprise between about 5 and about 50 wt-% ethylene. An FCC dry gas stream is a suitable dilute ethylene stream. Other dilute ethylene streams may also be utilized in the present invention such as coker dry gas streams. Because the present invention is particularly suited to FCC dry gas, the subject application will be described with respect to utilizing ethylene from an FCC dry gas stream.

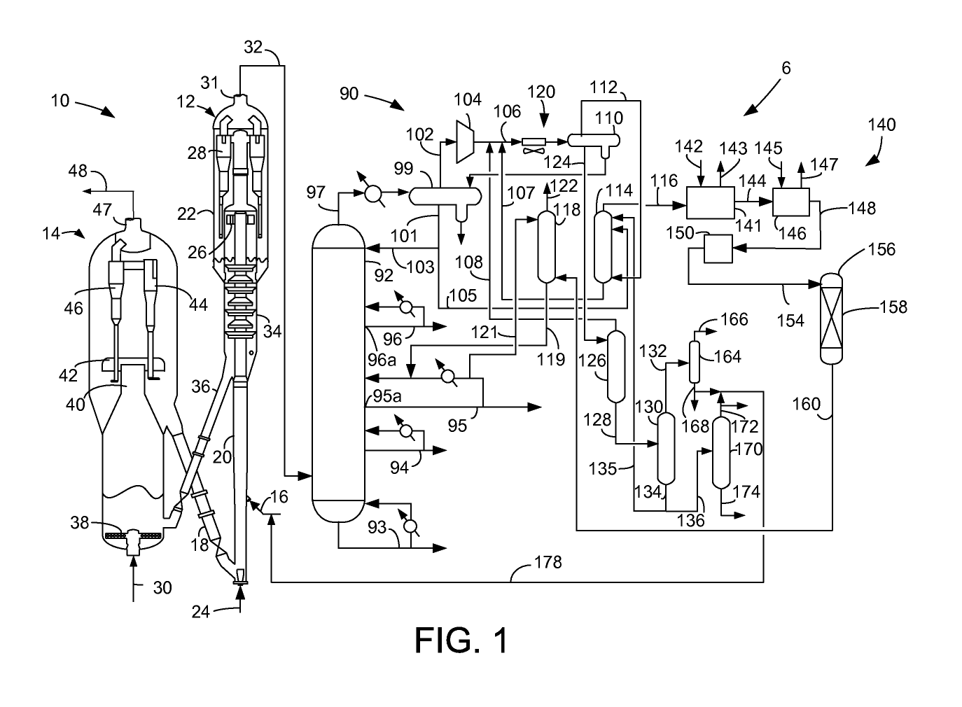

Now turning to FIG. 1, wherein like numerals designate like components, FIG. 1 illustrates a refinery complex 6 that generally includes an FCC unit section 10, a product recovery section 90 and a dry gas processing section 140. The FCC unit section 10 includes a reactor 12 and a catalyst regenerator 14. Process variables typically include a cracking reaction temperature of 400° to 600° C. and a catalyst regeneration temperature of 500° to 900° C. Both the cracking and regeneration occur at an absolute pressure below 506 kPa (72.5 psia).

FIG. 1 shows a typical FCC reactor 12 in which a heavy hydrocarbon feed or raw oil stream in a distributor 16 is contacted with a regenerated cracking catalyst entering from a regenerated catalyst standpipe 18. The FCC reactor 12 may be the only riser in the complex 6 or two or more FCC reactors may be utilized. Contacting in the FCC reactor 12 may occur in a narrow riser 20, extending upwardly to the bottom of a reactor vessel 22. The contacting of feed and catalyst is fluidized by gas from a fluidizing line 24. In an embodiment, heat from the catalyst vaporizes the hydrocarbon feed or oil, and the hydrocarbon feed is thereafter cracked to lighter molecular weight hydrocarbon products in the presence of the catalyst as both are transferred up the riser 20 into the reactor vessel 22. Inevitable side reactions occur in the riser 20 leaving coke deposits on the catalyst that lower catalyst activity. The cracked light hydrocarbon products are thereafter separated from the coked cracking catalyst using cyclonic separators which may include a primary separator 26 and one or two stages of cyclones 28 in the reactor vessel 22. Gaseous, cracked products exit the reactor vessel 22 through a product outlet 31 to line 32 for transport to a downstream product recovery section 90. The spent or coked catalyst requires regeneration for further use. Coked cracking catalyst, after separation from the gaseous product hydrocarbons, falls into a stripping section 34 where steam is injected through a nozzle to purge any residual hydrocarbon vapor. After the stripping operation, the coked catalyst is carried to the catalyst regenerator 14 through a spent catalyst standpipe 36.

FIG. 1 depicts a regenerator 14 known as a combustor, although other types of regenerators are suitable. In the catalyst regenerator 14, a stream of oxygen-containing gas, such as air, is introduced through an air distributor 38 to contact the coked catalyst. Coke is combusted from the coked catalyst to provide regenerated catalyst and flue gas. The catalyst regeneration process adds a substantial amount of heat to the catalyst, providing energy to offset the endothermic cracking reactions occurring in the reactor riser 20. Catalyst and air flow upwardly together along a combustor riser 40 located within the catalyst regenerator 14 and, after regeneration, are initially separated by discharge through a disengager 42. Additional recovery of the regenerated catalyst and flue gas exiting the disengager 42 is achieved using first and second stage separator cyclones 44, 46, respectively within the catalyst regenerator 14. Catalyst separated from flue gas dispenses through diplegs from cyclones 44, 46 while flue gas relatively lighter in catalyst sequentially exits cyclones 44, 46 and exits the regenerator vessel 14 through flue gas outlet 47 in flue gas line 48. Regenerated catalyst is carried back to the riser 20 through the regenerated catalyst standpipe 18. As a result of the coke burning, the flue gas vapors exiting at the top of the catalyst regenerator 14 in line 48 contain CO, CO₂, N₂ and H₂O, along with smaller amounts of other species. Hot flue gas exits the regenerator 14 through the flue gas outlet 47 in a line 48 for further processing.

The FCC product recovery section 90 is in downstream communication with the product outlet 31. In the product recovery section 90, the gaseous FCC product in line 32 is directed to a lower section of an FCC main fractionation column 92. The main fractionation column 92 is also in downstream communication with the product outlet 31. Several fractions of FCC product may be separated and taken from the main fractionation column including a heavy slurry oil from the bottoms in line 93, a heavy cycle oil stream in line 94, a light cycle oil in line 95 taken from outlet 95a and a heavy naphtha stream in line 96 taken from outlet 96a. Any or all of lines 93-96 may be cooled and pumped back to the main fractionation column 92 to cool the main fractionation column typically at a higher location. Gasoline and gaseous light hydrocarbons are removed in vapor line 97 from the main fractionation column 92 and condensed before entering a main column receiver 99. The main column receiver 99 is in downstream communication with the product outlet 31.

An aqueous stream is removed from a boot in the receiver 99. Moreover, a condensed light naphtha stream is removed in condensate line 101 while an overhead stream is removed in overhead line 102. The overhead stream in overhead line 102 contains gaseous light hydrocarbons which may comprise a dilute ethylene stream. A portion of the condensed stream in condensate line 101 is refluxed back to the main column in line 103, so the main fractionation column 92 is in upstream communication with the main column receiver 99. A net bottoms stream in bottoms line 105 and a net overhead stream in overhead line 102 may enter a gas recovery section 120 of the product recovery section 90.

The gas recovery section 120 is shown to be an absorption based system, but any gas recovery system may be used, including a cold box system. To obtain sufficient separation of light gas components the gaseous stream in overhead line 102 is compressed in compressor 104. More than one compressor stage may be used, and typically a dual stage compression is utilized to compress the gaseous stream in line 102 to between about 1.2 MPa to about 2.1 MPa (gauge) (180-300 psig) to provide a compressed first FCC product stream. Three stages of compression may be advantageous to provide additional pressure at least as high as 3.4 MPa (gauge) (500 psig).

The compressed light vaporous hydrocarbon stream in line 106 may be joined by streams in lines 107 and 108, cooled and delivered to a high pressure receiver 110. An aqueous stream from the receiver 110 may be routed to the main column receiver 99. A first FCC product stream comprising a gaseous hydrocarbon stream in line 112 from the overhead of the high pressure receiver 110 comprising the dilute ethylene stream is routed to a lower end of a primary absorber column 114. In the primary absorber column 114, the first FCC product stream is contacted with a second FCC product stream comprising unstabilized gasoline from the main column receiver 99 in bottoms line 105 directed to an upper end of the primary absorber column 114 to effect a separation between C₃+ and C₂− hydrocarbons. This separation is further improved by feeding stabilized gasoline from line 135 above the feed point of stream 105. The primary absorber column 114 is in downstream communication with an overhead line 102 of the main column receiver via lines 106 and 112 and the bottoms line 105 of the main column receiver 99. A liquid C₃+ bottoms stream in line 107 is returned to line 106 prior to cooling. A primary off-gas stream in line 116 from the primary absorber column 114 comprises the dilute ethylene stream which is a portion of the first FCC product stream for purposes of the present invention

The primary off-gas stream in line 116 enters the dry gas processing section 140. An advantage of the present invention is that the dilute ethylene stream in line 116 may undergo oligomerization without requiring further compression above operation pressure in the gas recovery section 120 of the product recovery section 90. However, a compressor may be utilized on line 116 if advantageous for product recovery and if compressor 104 has no third stage of compression.

The dilute ethylene stream of the present invention may comprise an FCC dry gas stream comprising between about 5 and about 50 wt-% ethylene and preferably about 10 to about 35 wt-% ethylene. Methane will typically be the predominant component in the dilute ethylene stream at a concentration of between about 25 and about 55 wt-% with ethane being substantially present at typically between about 5 and about 45 wt-%. Between about 1 and about 25 wt-% and typically about 5 to about 20 wt-% of hydrogen and nitrogen each may be present in the dilute ethylene stream. Saturation levels of water may also be present in the dilute ethylene stream. The dilute ethylene stream may have less than 3 wt-% and suitably less than 1 wt-% propylene and typically less than 25 wt-% and suitably less than 15 wt-% C3+ materials. Besides hydrogen, other impurities such as hydrogen sulfide, ammonia, carbon oxides and acetylene may also be present in the dilute ethylene stream.

We have found that many impurities in a dry gas ethylene stream can poison an oligomerization catalyst. Hydrogen and carbon monoxide can reduce the metal sites to inactivity. Carbon dioxide and ammonia can attack acid sites on the catalyst. Hydrogen sulfide can attack metals on a catalyst to produce metal sulfides. Acetylene can polymerize and produce gums on the catalyst or equipment.

A unit for removing impurities may be in communication between the primary absorber column 114 and an oligomerization reactor 156. The primary off-gas stream in line 116, comprising a dilute ethylene stream may be introduced into an optional amine absorber unit 141 to remove hydrogen sulfide to lower concentrations. A lean aqueous amine solution, such as comprising monoethanol amine or diethanol amine, is introduced via line 142 into absorber 141 and is contacted with the flowing primary off-gas stream to absorb hydrogen sulfide, and a rich aqueous amine absorption solution containing hydrogen sulfide is removed from absorption zone 141 via line 143 and recovered and perhaps further processed.

The amine-treated dilute ethylene stream in line 144 may be introduced into an optional water wash unit 146 to remove residual amine carried over from the amine absorber 141 and reduce the concentration of ammonia and carbon dioxide in the dilute ethylene stream in line 144. Water is introduced to the water wash in line 145. The water in line 145 is typically slightly acidified to enhance capture of basic molecules such as the amine. An amine-rich aqueous stream in line 147 and potentially rich in ammonia and carbon dioxide leaves the water wash unit 146 and may be further processed.

The optionally amine treated dilute ethylene and perhaps water washed stream in line 148 may then be treated in an optional guard bed 150 to remove one or more of the impurities such as carbon monoxide, hydrogen sulfide and ammonia down to lower concentrations. The guard bed 150 may contain an adsorbent to adsorb impurities such as hydrogen sulfide that may poison an oligomerization catalyst. The guard bed 150 may contain multiple adsorbents for adsorbing more than one type of impurity. A typical adsorbent for adsorbing hydrogen sulfide is ADS-12, for adsorbing carbon monoxide is ADS-106 and for adsorbing ammonia is UOP MOLSIV 3A all available from UOP, LLC. The adsorbents may be mixed in a single bed or can be arranged in successive beds.

A dilute ethylene stream in line 154 perhaps amine treated, perhaps water washed and perhaps adsorption treated to remove more hydrogen sulfide, ammonia and carbon monoxide will typically have at least one of the following impurity concentrations: about 0.05 wt-% and up to about 5.0 wt-% of carbon monoxide and/or about 0.1 wt-% and up to about 5.0 wt-% of carbon dioxide, and/or at least about 1 wppm and up to about 500 wppm hydrogen sulfide and/or at least about 1 and up to about 500 wppm ammonia, and/or at least about 5 and up to about 20 wt-% hydrogen. The type of impurities present and their concentrations will vary depending on the processing and origin of the dilute ethylene stream. With the exception of the impurities that have been removed, the dilute ethylene stream in line 154 will have the same or substantially the same composition as the dilute ethylene stream in line 116.

As explained, an advantage of the present invention is that the dilute ethylene stream in line 154 may undergo oligomerization without requiring further compression. However, a compressor may be utilized on line 154 if advantageous for oligomerization and perhaps if compressor 104 has no third stage of compression and if no compressor is on line 116. Heat exchangers and a heater (not shown) may be required to bring the compressed stream up to reaction temperature. The dilute ethylene stream is carried in line 154 to oligomerization reactor 156.

The oligomerization reactor 156 is in downstream communication with the primary absorber column 114. The oligomerization reactor 156 preferably contains a fixed catalyst bed 158, and the dilute ethylene feed stream contacts the catalyst preferably in a down flow operation. However, upflow operation may be suitable. The oligomerization reaction temperature may be in the range of 0-320° C., suitably 20 to 300° C. and preferably 80 to 150° C. which may be the temperature of the stream 154 without requiring additional heating. The oligomerization pressure may be between about 1.2 MPa to about 2.1 MPa (gauge) (180-300 psig). Higher pressure may be advantageous as high as 3.4 MPa (gauge) (500 psig). Gas space velocity may range between 1 and 5000 hr⁻¹.

The oligomerization catalyst preferably has a silica base with a metal from Group VIIIB in the periodic table using Chemical Abstracts Service notations. In an aspect, the silica base may include alumina. The base is preferably amorphous but may be crystalline such as a molecular sieve. AlMCM-41 and MCM-41 are suitable mesoporous base materials. In an aspect, the catalyst exhibits low acidity, having a silicon-to-aluminum ratio of no less than about 20 and preferably no less than about 50. Typically, the silicon and aluminum will only be in the base, so the silicon-to-aluminum ratio will be the same for the catalyst as for the base. The metals can either be impregnated onto or ion exchanged with the silica-alumina base. Co-mulling is also contemplated. Nickel is the preferred metal and in an aspect nickel (II) is preferred. Additionally, a suitable catalyst will have a surface area of between about 50 and about 500 m²/g as determined by nitrogen BET.

A suitable oligomerization catalyst of the present invention can be prepared according to J. Heveling, CATALYSTS AND CONDITIONS FOR THE HIGHLY EFFICIENT, SELECTIVE AND STABLE HETEROGENEOUS OLIGOMERIZATION OF ETHYLENE, Applied Catalysis A: General 173, 1 (1998). A synthesis of the ethylene oligomerization catalyst was carried out by the coprecipitation of freshly prepared sodium aluminate and commercially available sodium silicate solutions by the addition of nitric acid. A typical synthesis of the catalyst entails a first preparation of sodium aluminate solution. To 7.5 ml of distilled water, 4.5 g of Al(OH)₃ and 5.0 g of NaOH was added and placed in a flask equipped with a condenser. The mixture was allowed to react at reflux, with stirring, until a clear solution was obtained. Of distilled water, 250 ml was then added to the flask and the solution stirred and heated for a further minute. The second stage of the typical synthesis entails preparation of a silica-alumina hydrogel. To 199 ml of waterglass solution (Merck, 28% by mass SiO₂) and 1085 ml of distilled water, 228 ml of the hot sodium aluminate solution was added, followed by addition of 1.4 M nitric acid under vigorous stirring to obtain a gel with a pH of 9 within 1-2 min. The hydrogel was then aged at 25° C. for three days and then washed with distilled water until a neutral pH was obtained in the wash water. The third stage of the typical synthesis is the preparation of the solid silica-alumina. The diluted hydrogel obtained above was filtered using a Buchner funnel, to remove as much of the water as possible, and the more concentrated product then dried at 110° C. overnight followed by calcination at 550° C. for 3 hours. The Na⁺-form of the solid silica-alumina product with a silicon-to-aluminum ratio of 25 was thus obtained.

Nickel may be added to the silica-alumina solid support by ion exchange. Ion-exchange of the Na⁺-form of the solid support was effected by reflux with an aqueous solution of nickel chloride for 5 h using three moles of nickel(II) for every two moles of aluminum in the silica-alumina support. The green solids were then filtered and extensively washed with distilled water until the filtrates were free of chloride ions, otherwise detectable by the addition of silver nitrate. After drying at 110° C. and following acid digestion of the green solids, the catalyst contained 1.56% nickel by mass as determined by atomic absorption spectroscopy, an aluminum content of 1.6 mass-%, a sodium content of 0.68 mass-%, a BET surface area of 425 m²/g, an average pore radius of 18.7 Å, a pore volume of 0.75 cm³/g, and XRD analysis indicating an amorphous morphology. The catalyst may be activated by oxidation, but is not always necessary.

The oligomerization product may exit the oligomerization reactor 156 in line 160. To concentrate the unreacted ethylene, ethane and lighter gases and to recover heavier oligomers line 160 may be directed to a lower end of a secondary absorber column 118. The secondary absorber column is in downstream communication with the oligomerization reactor 156, and therefore the oligomerization reactor 156 is in communication between the primary absorber column 114 and the secondary absorber column 118. A circulating stream of light cycle oil in line 121 comprising a third FCC product stream diverted from line 95 to an upper end of the secondary absorber column 118 absorbs most of the C₅+ oligomers and some C₃-C₄ material in the oligomerization product stream. Other FCC product streams are contemplated to be a suitable third FCC product stream. The secondary absorber column 118 is in downstream communication with the main fractionation column 92, the primary absorber column 114 and the oligomerization reactor 156. Light cycle oil from the bottom of the secondary absorber column in bottoms line 119 richer in C₃+ material including oligomers is returned to the main fractionation column 92 via the pump-around for line 95. The main fractionation column 92 is in downstream communication with the secondary absorber column via bottoms line 119. Consequently, the main fractionation column 92 is in downstream and upstream communication with the oligomerization reactor 156. A secondary off-gas stream from the secondary absorber column 118 comprising dry gas of predominantly C₂− hydrocarbons with hydrogen sulfide, ammonia, carbon oxides and hydrogen is removed in overhead line 122 which can be further processed. Product oligomers are processed beginning in the main fractionation column 92 and recovered in the product recovery section 90. Both of the absorber columns 114 and 118 have no condenser or reboiler, but may employ pump-around cooling circuits.

In the high pressure receiver 110, the first FCC product stream exiting in line 112 is separated from a fourth liquid FCC product stream exiting from the bottom of the high pressure receiver in bottoms line 124. This fourth liquid FCC product stream, in bottoms line 124 including product oligomers, is sent to a stripper column 126 for fractionation. The stripper column 126 has no condenser but receives cooled liquid feed in line 124. Most of the C₂− material is removed in the overhead of the stripper column 126 and returned to line 106 via overhead line 108. A liquid bottoms stream from the stripper column 126 is sent to a debutanizer column 130 via bottoms line 128.

An overhead stream in overhead line 132 from the debutanizer column comprises C₃-C₄ olefinic product while a bottoms stream in line 134 comprising stabilized gasoline may be further treated and sent to gasoline storage. A portion of the stabilized gasoline in bottoms line 134 may be recycled in line 135 to a top of the primary absorber column above the inlet point of line 105 and 112 to improve the recovery of C₃+. The product in line 132 comprising C₃ and C₄ olefins may be used as feed for alkylation or subjected to further processing to recover olefins. In an aspect, line 132 is fed to an LPG splitter 164 to split a fifth FCC product stream of light olefins comprising C₃ olefinic material in the overhead line 166 from a C₄ olefinic bottoms stream in line 168.

In an embodiment, the net bottoms stream 136 from the debutanizer column may be fractionated in a naphtha splitter column 170. An overhead stream comprising C₅ to C₇ and optionally C₅ only, or C₅ to C₆ olefinic material in line 172 may be separated from a sixth FCC product stream of naphtha in bottoms stream in line 174. The sixth FCC product stream in line 174 may be further treated or sent to a gasoline tank for storage.

When a low-acidity oligomerization catalyst is employed in oligomerization reactor 156, lighter oligomers such as C₄ and C₆'s are produced. C₄ olefins may be useful in alkylation, in further oligomerization or in recycle to the FCC reactor or to an additional FCC reactor.

At least a portion of C₄ olefinic material in line 168 may be mixed with at least a portion of C₅ to C₇, optionally C₅ or C₅ to C₆ olefinic material in line 172 and at least a portion of which is recycled to the FCC reactor 12 via recycle line 178 as a seventh FCC product stream. Consequently, the recycle line 178 is in communication between the oligomerization reactor 156 and the FCC reactor 12. Alternatively, the seventh FCC product stream may be recycled to a separate FCC reactor (not shown). One or both of lines 172 or 168 should include a purge to prevent build up of paraffins if such a recycle is used.

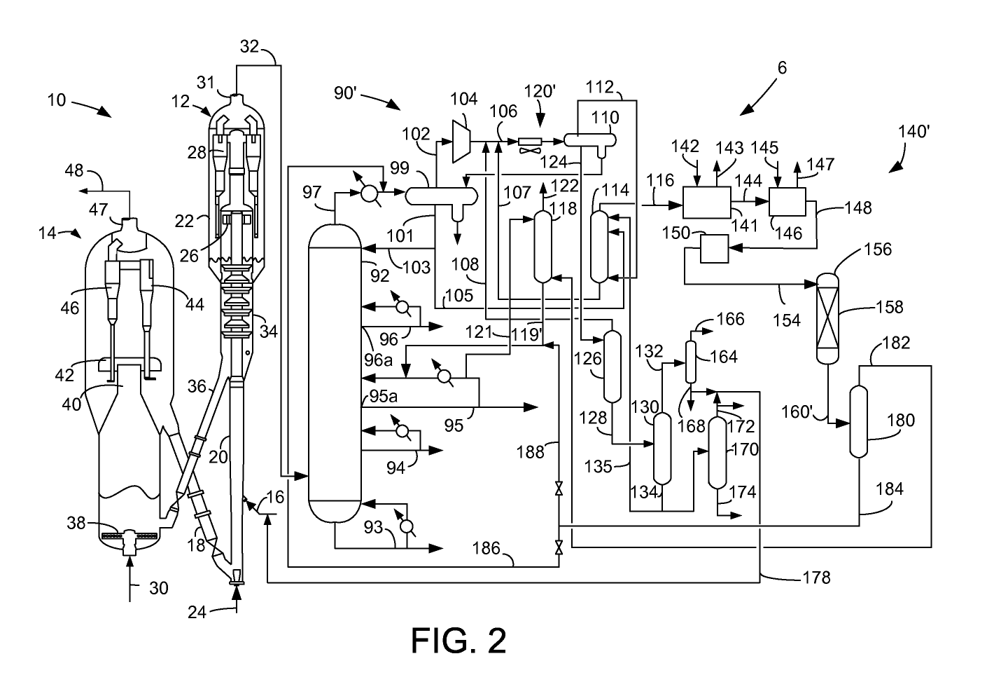

In an alternative embodiment, the oligomerization product stream is transported to an oligomerization separator 180. FIG. 2 shows this alternative embodiment. Elements in FIG. 2 that correspond to elements in but are different from FIG. 1 are indicated by a reference numeral with a prime sign (′). All other items in FIG. 2 are the same as in FIG. 1.

The gas recovery system 120′ and the dry gas processing section 140′ are different in FIG. 2 than in FIG. 1. The oligomerization product stream from the oligomerization reactor in line 160′ can be transported to an oligomerization separator 180 which may be a simple flash drum to separate a gaseous stream from a liquid stream. The oligomerization separator 180 is in downstream communication with the oligomerization reactor 156. The gaseous product stream in overhead line 182 from a top of the oligomerization separator 180 comprising light gases such as hydrogen, methane, ethane, unreacted olefins and light impurities may be transported to the secondary absorber column 118 in gas recovery section 120′ of the product separation section 90′. The secondary absorber column is in downstream communication via an overhead line 182 from the top of the oligomerization separator 180. The oligomerization separator 180 is in communication between the oligomerization reactor 156 and the secondary absorber column 118. The light gases in line 182 are processed in the secondary absorber column 118 and the gas recovery system 120′ just as described with respect to FIG. 1.

The liquid bottoms stream comprising heavier hydrocarbons including product oligomers in line 184 from the oligomerization separator 180 may be transported in line 186 regulated by a control valve thereon to join the line 97 to feed the main column overhead receiver 99. The unstabilized naphtha in line 186 requires stabilizing before recovery. If the liquid bottoms stream in line 184 has a relatively high concentration of heavy oligomers, it may alternatively be delivered in line 188 regulated by a control valve thereon to line 119′ to enter the main fractionation column 92 via pump around circuit 95.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A process comprising: contacting a first FCC product stream with a second FCC product stream to recover a dilute ethylene stream; contacting the dilute ethylene stream with an oligomerization catalyst to provide an oligomerization product comprising ethylene oligomers; and contacting the oligomerization product with a third FCC product stream.

2. The process of claim 1, wherein said FCC product streams are made by contacting cracking catalyst with a hydrocarbon feed stream to crack hydrocarbons to cracked product hydrocarbons having lower molecular weight and deposit coke on the cracking catalyst to provide coked cracking catalyst;
separating said coked cracking catalyst from said cracked products;
adding oxygen to said coked cracking catalyst;
combusting coke on said coked cracking catalyst with oxygen to regenerate said cracking catalyst;
separating said cracked products in a fractionation column.

3. The process of claim 1, wherein the dilute ethylene stream comprises between about 5 and about 50 wt-% ethylene.

4. The process of claim 1 wherein said oligomerization catalyst is a silica-alumina base with a silicon-to-aluminum ratio of at least 20.

5. The process of claim 4 wherein said oligomerization catalyst includes a metal from Group VIIIB in the periodic table.

6. The process of claim 5 wherein said oligomerization catalyst includes 0.5-15 wt-% nickel.

7. The process of claim 1 wherein said first FCC product stream is at least partially obtained from a vaporous overhead stream of a main fractionation column which is compressed to between about 1.2 MPa to about 2.1 MPa (gauge) (180-300 psig) to provide a compressed vaporous overhead stream.

8. The process of claim 7 wherein said compressed vaporous overhead stream is cooled and separated from a liquid fourth FCC product stream to provide said first FCC product stream before it is contacted with said second FCC product stream.

9. The process of claim 1 wherein said oligomerization product is separated and a overhead stream from a separator is contacted with the third FCC product stream.

10. The process of claim 8 further comprising fractionating said fourth FCC product stream to provide a fifth FCC product stream comprising light olefins, a sixth FCC product stream comprising naphtha and a seventh FCC product stream comprising light naphtha.

11. The process of claim 10 further comprising recycling at least a portion of the seventh FCC product stream to the cracking step.

12. A process comprising: taking a a portion of a first FCC product stream comprising between about 5 and about 50 wt-% ethylene and at least one impurity selected from the group consisting of at least about 0.05 wt-% carbon monoxide, at least about 1 wppm hydrogen sulfide, at least about 1 wppm ammonia, at least about 5 wt-% hydrogen and at least about 0.1 wt-% carbon dioxide; contacting said portion of said first FCC product stream with an oligomerization catalyst to provide an oligomerization product comprising ethylene oligomers and contacting the oligomerization product with another FCC product stream.

13. The process of claim 12 wherein said oligomerization catalyst is a silica-alumina base with a silicon-to-aluminum ratio of at least 20.

14. The process of claim 12 wherein said oligomerization catalyst includes 0.5-15 wt-% nickel.

15. The process of claim 12 wherein said first FCC product stream is obtained from a vaporous overhead of a main fractionation column which is compressed to between about 1.2 MPa to about 2.1 MPa (gauge) (180-300 psig) to provide a compressed first FCC product stream.

16. The process of claim 12 wherein said first FCC product stream is contacted with a second FCC product stream to provide said portion of said first FCC product stream.

17. The process of claim 12 wherein said contacting step is performed in a fixed bed of said oligomerization catalyst.

18. The process of claim 12 further comprising converting at least 40 wt-% of the ethylene in the portion of said first FCC product stream to heavier hydrocarbons.

19. A process comprising contacting a first FCC product stream with a second FCC product stream to recover a dilute ethylene stream; contacting the dilute ethylene stream with an oligomerization catalyst having a base with a silicon-to-aluminum ratio of at least 20 and including a Group VIIIB metal to provide an oligomerization product stream comprising ethylene oligomers and contacting the oligomerization product stream with a third FCC product stream to absorb ethylene oligomers and delivering said third FCC product stream and absorbed ethylene oligomers to an FCC main fractionation column.

20. The process of claim 19 wherein said first FCC product stream is obtained from a vaporous overhead of said main fractionation column which is compressed to between about 1.2 MPa to about 2.1 MPa (gauge) (180-300 psig) to provide a compressed first FCC product stream.
